(19) United States
(12) Patent Application Publication
(10) Pub. No.: US 2026/0193637 A1
(43) Pub. Date: Jul. 9, 2026

(54) HOUSING UNIT FOR TESTING A BIOLOGICAL SAMPLE

(71) Applicant: University of Rochester, Rochester, NY (US)

(72) Inventors: Jeffrey W. Beard, Rochester, NY (US); Alexander L. Evans, Rochester, NY (US)

(73) Assignee: University of Rochester, Rochester, NY (US)

(21) Appl. No.: 19/443,832

(22) Filed: Jan. 8, 2026

Related U.S. Application Data
(60) Provisional application No. 63/743,378, filed on Jan. 9, 2025.

(51) Int. Cl. C12N 15/10

(52) U.S. Cl. CPC C12N 15/1003

(57) ABSTRACT

A sample-testing system is disclosed. The system includes a separation system configured to separate a component of a sample for testing. The system further includes a housing enclosing the separation system. The housing includes a sample-receiving area configured to, when the housing is in a first configuration, receive the sample and direct the sample to the separation system, causing the separation system to separate the component of the sample. The housing further includes an extraction actuator configured to, when the housing is in a second configuration, extract the component of the sample from the separation system (and not extract the component when the housing is in the first configuration). The housing further includes a reconfiguration mechanism configured to reconfigure the housing from the first configuration to the second configuration.

### CROSS-REFERENCE TO RELATED APPLICATIONS

The present application claims priority under 35 U.S.C. § 119(e) to U.S. Provisional Patent Application No. 63/743,378, filed Jan. 9, 2025, which is incorporated by reference herein in its entirety.

### STATEMENT REGARDING FEDERALLY SPONSORED RESEARCH

This invention was made with government support under AI167035 awarded by the National Institutes of Health. The government has certain rights in the invention.

### FIELD

This disclosure relates generally to methods and systems of preparing a sample (e.g., a biological sample) for testing.

### BACKGROUND

Many blood-borne pathogens, such as HIV, require extraction and amplification of nucleic acids to diagnose or assess the disease state in a patient. The process of extracting the nucleic acids from whole blood is complex, requiring expensive laboratory equipment and highly trained technicians. This costly sample preparation not only limits these critical tests to high-end laboratories, but also repeatedly exposes technicians and clinicians to the potentially infectious sample. This document describes methods and systems that address issues such as those discussed above, and/or other issues.

### SUMMARY

The present disclosure describes embodiments related to testing samples, such as biological samples. In an embodiment, a sample-testing system includes a separation system configured to separate a component of a sample for testing. The system further includes a housing enclosing the separation system. The housing includes a sample-receiving area configured to, when the housing is in a first configuration, receive the sample and direct the sample to the separation system, causing the separation system to separate the component of the sample. The housing further includes an extraction actuator configured to, when the housing is in a second configuration, extract the component of the sample from the separation system and when the housing is in the first configuration, the extraction actuator is configured to not extract the component of the sample. The housing further includes a reconfiguration mechanism configured to reconfigure the housing from the first configuration to the second configuration.

Implementations of the disclosure may include one or more of the following optional features. In some examples, when the housing is in the second configuration, the reconfiguration mechanism is inoperable. The reconfiguration mechanism may include a pull tab. The pull tab may include a locking mechanism that prevents the pull tab from reconfiguring the housing when the housing is in the second configuration. The locking mechanism may include a ratchet. In some examples, the extraction actuator is configured to be inoperable after the component of the sample is extracted. The extraction actuator may be a pushbutton configured for a single use. In some examples, the separation system includes a capture material and the extraction actuator is configured to extract the component of the sample from the separation system by extracting some or all of the capture material. The reconfiguration mechanism may be configured to align the some or all of the capture material with the extraction actuator when the housing is in the second configuration. In some examples, the extraction actuator is not affixed to the reconfiguration mechanism. The housing may further include a wash-receiving area configured to, when the housing is in the first configuration, receive a wash substance and direct the wash substance to the separation system. The sample-receiving area may include a basin. In some examples, the interior of the housing defines a containment chamber configured to collect unwanted components of the sample and/or the wash substance.

In an embodiment, a method includes receiving the system of claim 1 in the first configuration, adding the sample to the sample-receiving area, reconfiguring the system into the second configuration, and operating the extraction actuator, causing the component of the sample to be extracted.

Implementations of the disclosure may include one or more of the following optional features. In some examples, the method further includes before reconfiguring the system into the second configuration, adding a wash substance to a wash-receiving area. The method may further include testing the component of the sample.

In an embodiment, a kit includes (i) a system of claim 1; (ii) a collector configured to collect the sample; and (iii) a container configured to receive the component. Implementations of the disclosure may include one or more of the following optional features. In some examples, the kit further comprises one or more of a washing buffer, a nucleic acid probe, and a reaction solution.

Implementations of the disclosure may include one or more of the following optional features. In some examples, the kit further comprises one or more of a washing buffer, a nucleic acid probe, and a reaction solution. In some examples of the system, method, or kit embodiments, the sample-receiving area is configured to receive a blood sample, a plasma sample, a serum sample, a sputum sample, a urine sample, a saliva sample, a buccal mouthwash sample, a tissue sample, fecal matter, sweat, spinal fluid, amniotic fluid, interstitial fluid, tear fluid, or bone marrow.

In an embodiment, a sample-testing system includes a separation system configured to separate a component of a sample for testing. The system further includes a housing enclosing the separation system. The housing includes a sample-receiving area configured to, when the housing is in a first configuration, receive the sample and direct the sample to the separation system, causing the separation system to separate the component of the sample. The housing further includes a reconfiguration mechanism operable to reconfigure the housing from the first configuration to a second configuration. The sample-testing system further includes an assay system configured to, when the housing is in the second configuration, receive the component of the sample, and perform an assay on the component of the sample.

In a further aspect, the disclosure provides a housing as described herein.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 shows an example sample-testing system.

FIG. 2 shows an upper portion of a housing according to an embodiment.

FIG. 3 shows a lower portion of a housing according to an embodiment.

FIG. 4 shows a pull tab according to an embodiment.

FIG. 5 shows an exploded view of the example sample-testing system.

FIGS. 6A-C show cutaway views of the example sample-testing system.

FIGS. 7A-C show partial views of the example sample-testing system.

FIG. 8 shows an exploded view of another example sample-testing system.

FIG. 9 shows a flowchart of a method.

In the drawings, like reference numbers generally indicate identical or similar elements. Additionally, generally, the left-most digit(s) of a reference number identifies the drawing in which the reference number first appears.

### DETAILED DESCRIPTION

As used in this document, the singular forms “a,” “an,” and “the” include plural references unless the context clearly dictates otherwise. Unless defined otherwise, all technical and scientific terms used herein have the same meaning(s) as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

In this document, when terms such as “first” and “second” are used to modify a noun or phrase, such use is simply intended to distinguish one item from another and is not intended to require a sequential order unless specifically stated. The term “about” when used in connection with a numeric value, is intended to include values that are close to, but not exactly, the number. For example, in some embodiments, the term “about” may include values that are within +/−10 percent of the value.

The present disclosure relates generally to methods and systems of preparing a sample (e.g., a biological sample) for testing. In particular, the present disclosure relates to workflow-enhancing housing units for sample-preparation and/or testing systems. Example sample-preparation systems that are suitable for use within the housing are disclosed in PCT application PCT/US2022/034636, titled “Devices and Methods for isolating and detecting viral nucleic acids,” which is hereby incorporated by reference in its entirety. Other sample preparation/isolation/testing systems are also within the scope of this disclosure. The disclosed housing units enhance sample-preparation systems by including features that support safe, convenient, and tamper-resistant preparation and/or testing of samples, e.g., biological samples such as blood or plasma.

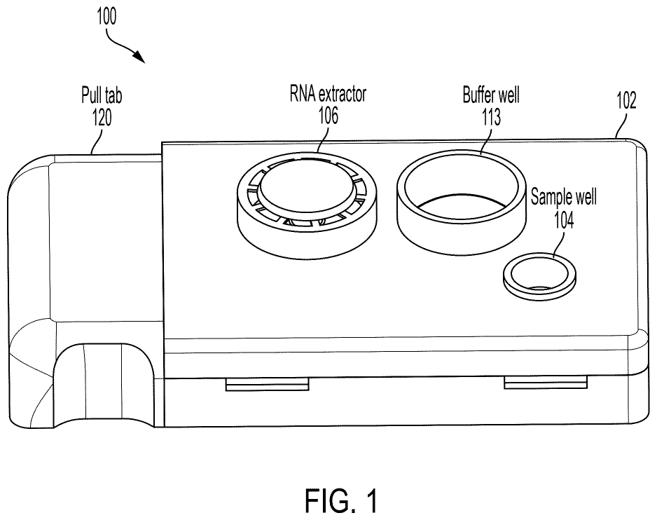

FIG. 1 shows an example sample-testing system 100. The system 100 includes a housing 102 enclosing components of a sample preparation/isolation/testing system. Thus, during normal operation of the system 100, the user does not come into direct contact with the enclosed sample preparation/isolation/testing system. Instead, the user provides an appropriate sample, which is automatically processed and prepared for testing. The housing 102 includes features which facilitate this process. For example, the housing includes a sample-receiving area 104 configured to receive a sample, e.g., a biological sample, such as blood or plasma, or the like for testing. The sample-receiving area 104 may be configured to receive other biological samples, including, but not limited to, urine, saliva, mucus, and so forth. In some examples, the sample-receiving area 104 is configured to receive other substances for testing, such as drinking water. The sample-receiving area 104 may be a basin or a recessed area within the housing 102 configured to contain a testable amount of the sample. In some examples, a testable amount of a sample, such as whole blood, may be 65-100 microliters.

As shown, the sample-receiving area 104 is shaped like a well, having vertical walls to contain the sample, and a small lip. The sample-receiving area 104 may include an aperture, e.g., at the bottom of the recessed area, which is configured to direct some or all of the sample to a suitable portion of the sample preparation/isolation/testing system within the housing 102. The sample-receiving area 104 may be sized and/or shaped to contain the sample at least during the period of time that the sample is being directed to the sample preparation/isolation/testing system. In the case of hazardous samples (e.g., biological samples that may contain harmful pathogens), the well may provide some protection against contact with the sample. For example, the sample-receiving area 104 may have a relatively narrow upper opening, helping to avoid accidental contact with the sample by, e.g., a finger of a user.

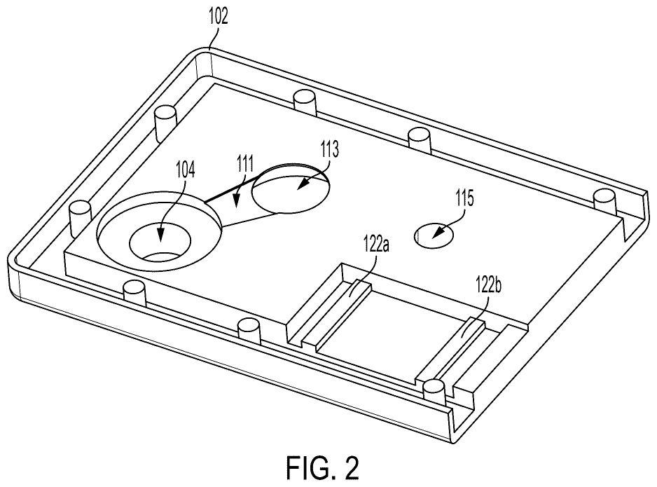

FIG. 2 shows the upper portion of the housing 102 of FIG. 1, as seen from below. This view provides additional details on certain features. For example, a channel 111 extends between the aperture at the bottom of the sample-receiving area 104 and an aperture at the bottom of a wash-receiving area 113 (described in more detail below). This view also shows stops 122a, 122b which extend downward from a roof portion of the housing 102. These stops 122 will also be described in more detail below. This view also shows another aperture (or hole 115) which will be described with respect to the extraction actuator 106.

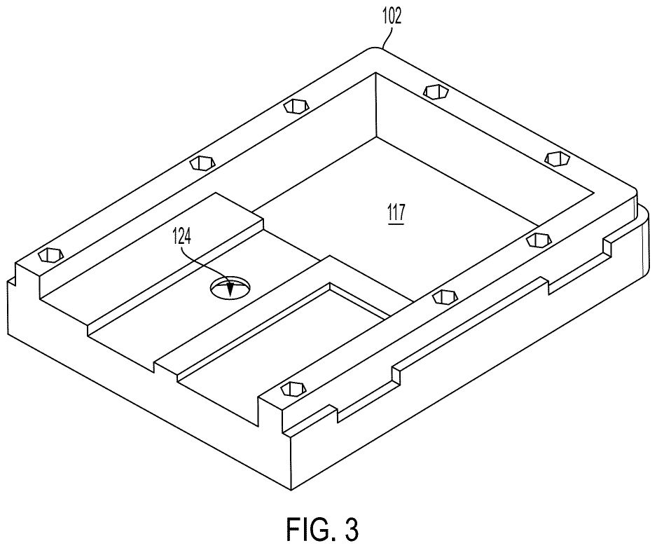

FIG. 3 shows the lower portion of the housing 102 of FIG. 1. In some embodiments, the upper portion (shown in FIG. 2) and the lower portion (shown in FIG. 3) are formed separately, e.g., for ease of manufacture, and are mated together during final assembly of the system 100. Separately manufacturing the lower portion and the upper portion may facilitate forming the features of the housing 102 including, but not limited to, the various apertures described above. As shown in FIG. 3, the lower portion includes a passageway 124, through which the processed sample may be extracted from the system 100 for testing via a separate assay device. In other embodiments, the passageway 124 leads to an internal assay system or device. Also as shown, the lower portion of the housing 102 includes a containment chamber 117 configured to receive unwanted substances that may result from processing the sample. That is, the system 100 may protect the user from exposure to potentially hazardous substances by containing all the products of sample processing (other than the processed sample itself) within an impervious chamber.

As discussed above, the system 100 also includes a sample preparation/isolation/testing system enclosed within the housing 102. That is, the housing 102 may enclose a system configured to receive the sample from the sample-receiving area 104 and process the sample for testing. In some examples, processing includes isolating one or more components of the sample for testing. Isolating components may include separating genetic material from the sample, e.g., separating and/or isolating nucleic acids, such as DNA or RNA. In some examples, isolating components includes separating and/or isolating proteins or other molecules.

In some examples, the preparation/isolation/testing system includes multiple layers. The multiple layers may each perform a specific function. For example, a first layer (e.g., adjacent to the aperture at the bottom of the sample-receiving area 104) may include a filter, such as a fibrous matrix configured to capture, e.g., leukocytes from a whole blood sample. The remaining components (e.g., plasma and red blood cells) may pass through the first layer to a second layer. The second layer may be treated with a reagent to further process the sample. In some examples the reagent is selected for its ability to lyse cells. Example lysis reagents include Triton X-100. The second layer may be configured to wick the plasma from the first layer. For example, the second layer may wick plasma through material that occupies the channel 111 that extends between the sample-receiving area 104 and the wash-receiving area 113. The second layer may be a lysis-reagent layer (also known as a plasma lysing layer) including a material that has been infused with a lysis reagent. The plasma lysing layer may be formed of paper, polymer, fiberglass, synthetic fiber, a combination of these or other materials that effectively provide for the second layer to wick the plasma from the first layer and to apply the reagent (e.g., lysis reagent) to the plasma. Thus, the processed sample is wicked toward the aperture beneath the optional wash-receiving area 113. Some or all of the second layer may be configured to capture the processed sample for testing. In some examples, a separate, small amount of material, known as a capture pad, may be located beneath the second layer and configured to capture the processed sample for testing. In these embodiments, the separate capture pad may be extracted from the system 100 for testing. In some embodiments, the capture pad may be a nucleic acid absorption pad, e.g., a small disc (about 2 mm to 5 mm in diameter) of commercially available filter paper (e.g., Whatman Fusion 5, Cytivia) that has been functionalized with a polysaccharide such as chitosan. As the plasma solution is wicked through the second layer, the chitosan may become protonated. The negatively charged genetic material (e.g., viral RNA), may become electrostatically adsorbed to the capture pad while the bulk solution passes through.

In some examples, the preparation/isolation/testing system includes additional layers. For example, a lowest layer of the preparation/isolation/testing system (e.g., below the capture pad) may be a containment layer (e.g., in the containment chamber 117) configured to absorb liquids that pass through upper layers. That is, the containment layer may be configured to receive the products of processing the sample for testing, collecting the unwanted liquids in a hydrophilic material, and retaining the liquids within the containment chamber 117. In some examples, an impermeable plastic layer may generally separate the containment layer from the remainder of the housing 102 except where necessary (e.g., where liquids enter the containment layer, such as directly beneath the wash-receiving area 113) to assist in retaining liquids within the wicking layer.

Referring back to FIG. 2, the housing 102 may include a wash-receiving area 113. The wash-receiving area 113 may be configured to receive one or more wash substances, such as a solvent or water, e.g., to remove impurities or unwanted materials from the sample (e.g., after the lysing process). In some examples, the wash-receiving area 113 is configured to direct the wash substance(s) to the preparation/isolation/testing system, e.g., to further process the sample after the filtration and lysis steps. In one example, a first wash substance is configured to prime the capture pad for capturing genetic material, such as viral RNA. The capture pad may be advantageously positioned directly beneath the wash-receiving area 113 for receiving the wash substance(s). A second wash substance may be configured to remove remaining lysed components from the capture pad (e.g., flushing those components into a wicking layer positioned beneath the capture pad, optionally through a strategically placed aperture in a plastic layer above the wicking layer). That is, the aperture may be placed beneath the wash-receiving area 113 and may be the only aperture in the plastic layer so as to help retain the unwanted components in the wicking layer (e.g., avoiding the unwanted components from working their way back to the capture pad). Other embodiments may exclude the plastic layer.

The wash-receiving area 113 itself may have physical characteristics that are somewhat similar to the sample-receiving area 104. That is, the wash-receiving area 113 may be a recessed area configured to receive a suitable amount of wash substances. In one example, each application of a wash substance includes 500 microliters of one or more wash substances. In this example, the wash-receiving area 113 may be larger than the sample-receiving area 104 due to the requirement for containing a larger volume of liquid. Furthermore, if the wash-receiving area 113 is only expected to receive non-hazardous materials, the wash-receiving area 113 may not pose the same risk to the user from accidental contact as the sample-receiving area 104. Therefore, the wash-receiving area 113 may not have the same contact-preventing features as the sample-receiving area 104. For example, the wash-receiving area 113 may have a relatively wide opening that may not prevent accidental contact of, e.g., a finger of the user with the wash substance(s).

The housing 102 may also include an actuator 106 (e.g., an extraction actuator 106). The actuator 106 may be configured to extract the component of the sample from the separation system. For example, the actuator 106 may be configured to extract some or all of the capture layer/capture pad from the sample-testing system 100. The extracted (portion of the) capture pad may then be tested, e.g., for the presence of genetic material of interest. In some examples, the extracted capture pad (containing components of the sample) is transferred to a loop-mediated isothermal amplification (LAMP) to detect the genetic material of interest. In other examples, the extracted capture pad is transferred to a PCR testing system or other assay device. In some embodiments, the sample-testing system 100 includes an integrated (e.g., internal) assay system. In these embodiments, the actuator 106 may be configured to transfer the (portion of the) capture pad to the integrated assay system, rather than extracting the capture pad for transfer to an external assay device.

As shown, the actuator 106 includes a pushbutton configured for a single use. That is, the extraction actuator 106 may be configured to be inoperable after the capture pad (including the component of the sample) is extracted. In the illustrated embodiment, the pushbutton is generally held in place by thin breakaway tabs which are configured to yield under sufficient pressure. That is, in order to depress the pushbutton, the user may have to apply at least a threshold amount of pressure to cause the tabs to break away. As shown, the pushbutton, once depressed, will remain in that state. That is, the pushbutton does not include a spring or other biasing mechanism that would cause the pushbutton to return to its initial position after pressure is removed. Furthermore, the pushbutton may include an engagement feature that secures the pushbutton in the depressed position. For example, the pushbutton may include a shaft that extends into the housing 102 (at least when the pushbutton has been depressed), e.g., extending through a hole 115 in the housing 102 beneath the pushbutton. The interface between the shaft and the housing 102 may create an interference fit such that, unless the system 100 includes a biasing mechanism or other form of pressure from below, the pushbutton will remain in the depressed position. The interference fit may be enhanced by increasing the diameter of the shaft at or near where the shaft passes through the hole 115 when the pushbutton is in the depressed position.

Other actuator 106 embodiments may omit breakaway tabs in favor of an engagement feature as described above. That is, the engagement mechanism (possibly enhanced as described above and/or enhanced with a ratchet mechanism) may be sufficient to secure the single-use pushbutton 106 in the depressed position. These embodiments may include a spring or biasing mechanism to help support the pushbutton before it is depressed. Other embodiments of single-use pushbuttons are also within the scope of this disclosure.

The housing 102 may include multiple configurations. As shown, the housing 102 is in a first configuration. In this configuration, the sample-receiving area 104 is configured to receive the sample and direct the sample, e.g., to the separation system within the housing 102, as described above. Directing the sample to the separation system causes the separation system to separate a component of the sample for testing, as described above. However, in this first configuration, the actuator 106 may be inoperable. That is, the actuator 106 may be precluded from extracting the processed sample for testing (or from transferring the processed sample to an integrated assay system). To make the actuator 106 operable, it may be necessary to reconfigure the housing 102 first. That is, the housing 102 may be reconfigurable from a sample-receiving configuration, in which the sample-testing system 100 is configured to receive the sample (and, optionally receive one or more wash substances), and a sample-extraction configuration, in which the capture pad (including the separated/isolated genetic material) is extracted for transfer to an assay device (either external to, or integrated with, the sample-testing system 100).

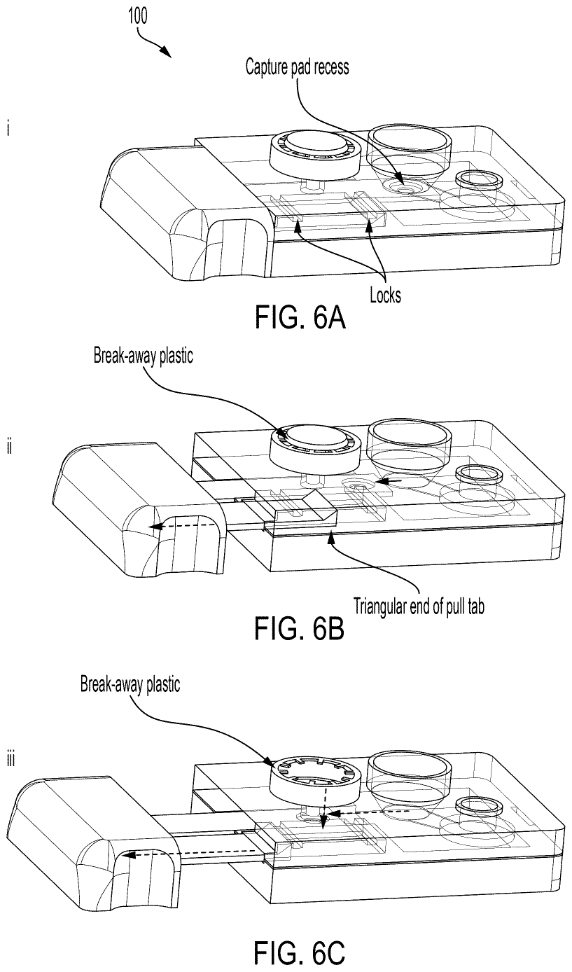

As described in more detail below with respect to FIGS. 6A-C and 7A-C, a reason for designing the housing 102 to be reconfigurable is to allow the capture pad to be physically translated (e.g., moved horizontally) from a first position (e.g., between the wash-receiving area 113 and the wicking layer), which may be advantageous during the process of receiving the sample and isolating components for testing, to a second position (e.g., beneath the extraction actuator 106) which may be more advantageous for extracting the capture pad (and isolated components of the sample). For this reason, the extraction actuator 106 may be rendered inoperable when the housing 102 is configured to receive the sample and may only be operable once the housing 102 has been reconfigured for extraction. This form of interlocking may enhance the workflow and/or operation of the sample-testing system 100. That is, the extraction actuator 106 may be effectively disabled until the capture pad has been physically translated to its extraction position. Furthermore, a single-use pushbutton 106 may provide a clear indication that the processed sample has been extracted. Thus, the feature of a single-use pushbutton 106 may also enhance the workflow and/or operation of the sample-testing system 100.

As shown, the housing 102 includes a reconfiguration mechanism 120 in the form of a pull tab 120. The pull tab 120 may include an ergonomic shape to enhance grip and/or comfort during operation. The pull tab 120 may be initially fully inserted in the housing 102. That is, the sample-testing system 100 may be in the sample-receiving configuration when the pull tab 120 is fully inserted. As described above, in this first configuration, the actuator 106 may be inoperable. To reconfigure the sample-testing system 100, the user may operate the reconfiguration mechanism 120 (e.g., pull the pull tab 120 so that it slides at least partially away from the housing 102). In some examples, when the pull tab 120 is fully extended, the sample-testing system 100 is in the second configuration (e.g., the sample-extraction configuration). When the sample-testing system 100 is in the second configuration, the actuator 106 may be operable to extract the capture pad for testing.

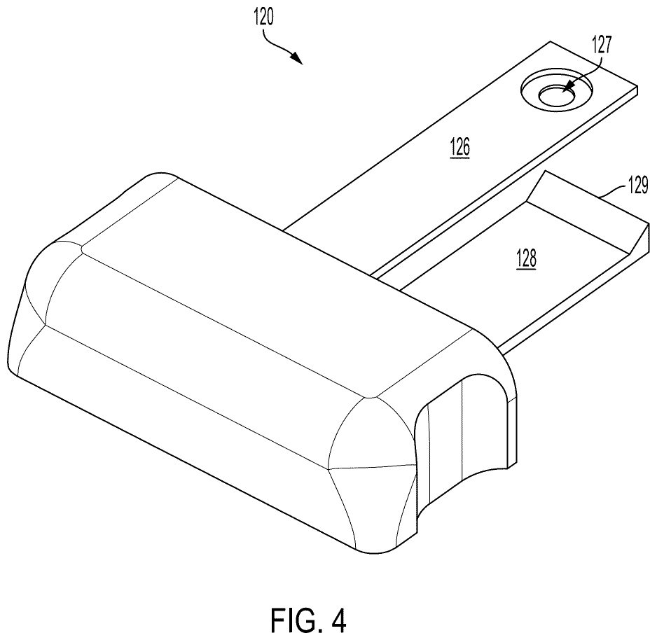

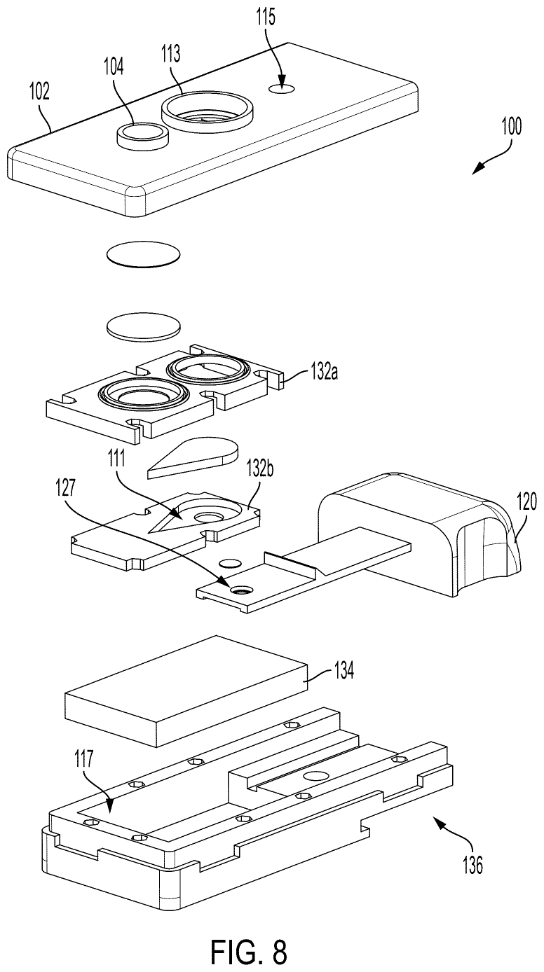

Referring to FIG. 4, details of an example pull tab 120 are shown. The pull tab 120 includes a first mechanical arm 126 which includes a recessed area 127 where the capture pad may be located. In this configuration, the capture pad translates (e.g., moves horizontally) with the pull tab 120 as the pull tab 120 is pulled out. The pull tab 120 also includes a second mechanical arm 128. The second mechanical arm 128 includes at least one upward protrusion 129 configured to engage with the downward-protruding stops 122 shown in FIG. 2. In some examples, the functionality of the first and second mechanical arms may be combined into one single arm (e.g., the example embodiment shown in FIG. 8). That is, the single mechanical arm may both (i) cause the capture pad to translate from a sample-receiving position to an extraction position and (ii) the arm may engage with one or more stops 122 as the pull tab 120 is pulled out.

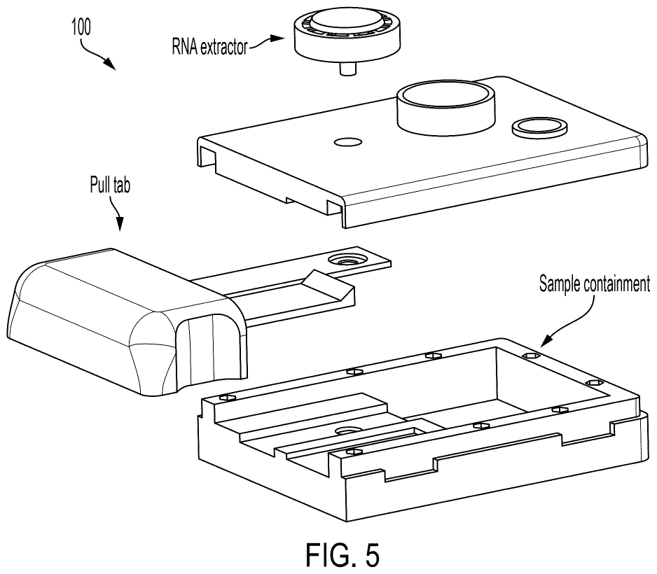

FIG. 5 shows an exploded view of the example sample-testing system 100. In particular, the exploded view shows separate components included in the housing 102. These components include the upper portion of FIG. 2, the lower portion of FIG. 3, and the pull tab 120 of FIG. 4. During final assembly, the components of the preparation/isolation/testing system (e.g., the wicking layer, capture pad, and so on) may be installed within the housing 102, before the upper portion and the lower portion are mated together. Once the upper and lower portions are mated (e.g., snapped together), the containment chamber 117 (that collects the unwanted processing products) may be sealed from the outside environment.

The components in FIG. 5 may be individually manufactured using extrusion molding, 3D printing, or other suitable technique. The materials used to manufacture these components may be lightweight polymers such as thermoplastic materials that are resistant to chemicals. Examples of suitable polymers include polyethylene and polypropylene. The components may also be pressed or stamped from metal stock, such as sheets. In some examples, different components are manufactured using different materials and/or different manufacturing techniques.

Referring generally to FIGS. 6A-C, cutaway views of the example sample-testing system 100 are shown. In particular, the cutaway views show the operation of the pull tab 120 in more detail. In particular, the cutaway views illustrate the interaction between the pull tab 120 and the actuator 106. FIG. 6A shows the housing 102 in the first (sample-receiving) configuration. In this configuration, the pull tab 120 is fully inserted within the housing 102. In this configuration, the capture pad is located beneath the wash-receiving area 113. As shown, the capture pad is located within a recessed area 127 of a mechanical arm 126 that may be a component of (or may be affixed to) the pull tab 120, so that the capture pad translates toward its extraction position as the pull tab 120 is pulled.

FIG. 6B shows the housing 102 in transition between the sample-receiving configuration and the sample extraction configuration. That is, the pull tab 120 has been partially pulled out. In this transition state, the mechanical arm renders the extraction actuator 106 inoperable. That is, a shaft that extends downward from the extraction actuator 106, is blocked by the first mechanical arm 126, which is located beneath the extraction actuator 106. Because the mechanical arm 126 is solid along most of its length, the shaft is prevented from downward motion. As can also be seen in FIG. 6B, the housing 102 includes one or more locking features, such as mechanical stops 122, which engage with the pull tab 120 at particular positions of the pull tab 120. As shown, the pull tab 120 includes a second mechanical arm 128 configured to engage the one or more stop(s) 122. In some examples, the functionality of the first and second mechanical arms may be combined into one arm. That is, the mechanical arm may (i) cause the capture pad to translate from a sample-receiving position to an extraction position and (ii) engage with one or more stops 122 as the pull tab 120 is pulled.

In the illustrated configuration, stops 122 protrude downward from a roof portion of the housing 102 above the second mechanical arm 128. The second mechanical arm 128 includes at least one upward protrusion 129 configured to engage with the downward-protruding stops 122. Here, the second mechanical arm 128 includes a wedge-shaped upward protrusion 129 which acts like a ratchet. That is, once the wedge shape passes under the stop 122, the stop 122 will prevent the protrusion 129 from passing back underneath the stop 122. Thus, once the pull tab 120 is pulled out a threshold distance, it will be difficult or impossible to push the pull tab 120 back to its original position. Instead, the user will have to continue to pull the pull tab 120 until the housing 102 is in the extraction configuration.

Although the wedge-shaped upward protrusion 129 allows the pull tab 120 to be pulled outward past each stop 122, it may require some effort to do so. That is, the protrusion 129 may be at least partially compressed as it passes under a stop 122. Furthermore, the protrusion 129 may be biased upward, e.g., by a spring or by the mechanical arm 128 itself. Thus, as the pull tab 120 is pulled, the force of the upward bias on the protrusion 129 and/or the compression force on the protrusion 129 result in greater mechanical force required to pull the pull tab 120. The increased mechanical force required to pull the pull tab 120 past a stop 122 may be used advantageously to help retain the pull tab 120 in its initial position (which may also help retain the capture pad in an optimal position for sample isolation/separation/capture).

As shown, the housing 102 includes two stops 122. A first stop 122a (near the beginning of travel of the second mechanical arm 128) helps to retain the pull tab 120 in its original position (and, thus, the housing 102 in a sample-receiving configuration). A second stop 122b (near the end of travel of the second mechanical arm 128) helps to lock the pull tab 120 in a fully extended position (and, thus, the housing 102 in a sample-extracting configuration). Furthermore, the second stop 122b serves to lock the pull tab 120 in the fully extended configuration. Locking the pull tab 120 in this way serves to accurately position the capture pad beneath the extraction actuator 106, facilitating the extraction process.

FIG. 6C shows the pull tab 120 fully extended and locked in position. The housing 102 is in the sample-extraction configuration, with the capture pad directly beneath the extraction actuator 106. Furthermore, the recessed area 127 in the mechanical arm 126 where the capture pad is located has a hole in its bottom. In this configuration, the shaft of the extraction actuator 106 may pass through the hole. Thus, in this configuration, the extraction actuator 106 is operable to extract the capture pad through the hole in the bottom of the recessed area 127. In some examples, the recessed area 127 is circular with a diameter of about 4 mm, and the hole in the base of the recessed area 127 is circular with a diameter of about 2 mm. The capture pad may have sufficient rigidity or be otherwise configured to remain in the recessed area 127 until pushed through the 2 mm hole by the shaft of the extraction actuator 106. The shaft may be somewhat less than 2 mm in diameter so that the capture pad can easily pass through the 2 mm hole. In some examples, when the housing 102 is in the sample-extraction configuration, the capture pad is located above a passageway 124 in the housing 102. In this position, the extracted capture pad may simply fall through the passageway 124 and outside of the housing 102, where it will be available for testing. In other configurations, the extracted capture pad falls into a sample-collection region of the housing 102, rather than falling through the bottom of the housing 102. In some examples (e.g., the example embodiment shown in FIG. 8), the extracted capture pad falls into a sample-collection region that can be removed from the housing to provide access to the extracted capture pad for testing. In an embodiments that include an integrated assay device, the assay device may be located beneath the location of the capture pad, so that the extracted capture pad falls into the assay device for testing.

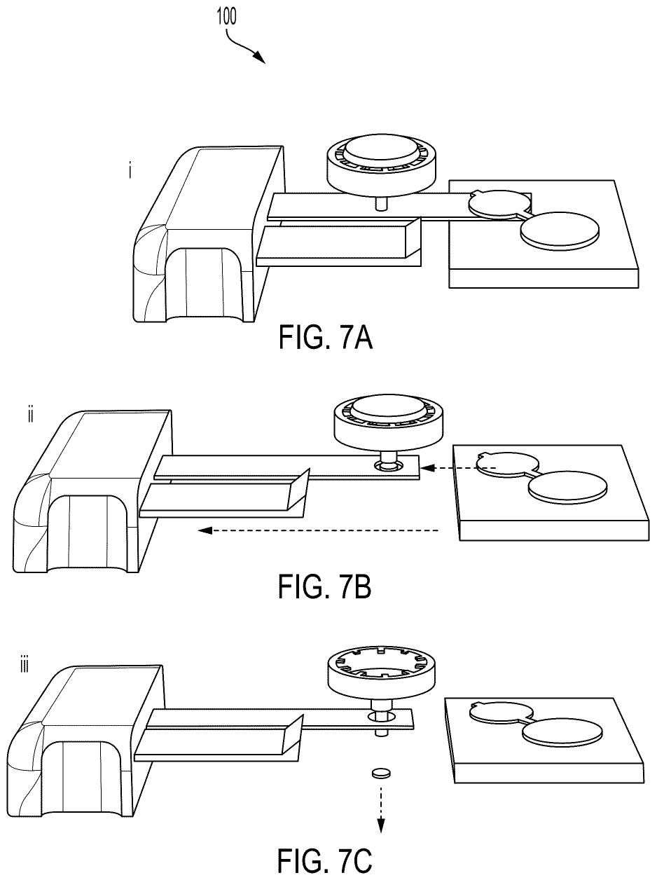

Referring generally to FIGS. 7A-C, views of the example sample-testing system 100 excluding the upper and lower portions of the housing 102 are shown. In particular, the cutaway views show the operation of the interaction between the pull tab 120 and the sample isolation/separation/capture system in more detail. Each of FIGS. 7A-C roughly corresponds with FIGS. 6A-C. For example, FIG. 7A shows the sample-receiving configuration, with the pull tab 120 fully inserted. In this configuration, the extraction actuator 106 is inoperable due to interference from the first mechanical arm 126. That is, downward movement of the shaft of the actuator 106 is blocked by the first mechanical arm 126. The capture pad is located within a recessed area 127 of the first mechanical arm 126. In this configuration, the capture pad is beneath the wash-receiving area 113. In FIG. 7A, the capture pad cannot be seen because it is beneath an upper layer of the sample isolation/processing system.

In FIG. 7B, the pull tab 120 is fully extended (e.g., somewhat further than in roughly corresponding FIG. 6B). Here, the recessed area 127 and capture pad are visible near the extraction actuator. That is, the capture pad has been translated due to the operation of the pull tab 120. In other embodiments, the capture pad may remain stationary, and other components may translate due to operation of the pull tab 120. For example, the wash-receiving area 113 (and associated portions of the isolation/separation/capture system may translate away from the capture pad and/or the extraction actuator 106 may translate over the capture pad. Regardless of what components are translated by the pull tab 120, when the extraction actuator 106 is positioned above the capture pad, the extraction actuator 106 may be operable to extract the extraction pad (and the associated processed sample).

FIG. 7C shows the capture pad after extraction by the extraction actuator. As shown, a shaft of the actuator 106 is extending through the recessed area 127 of the first mechanical arm. That is, the hole in the bottom of the recessed area 127 allows the shaft to have sufficient downward motion as to force the extraction pad through the hole (and, e.g., through the associated passageway 124).

FIG. 8 shows an exploded view of another example sample-testing system. In this example, the overall device footprint is about the size of a lateral flow assay, such as a SARS-CoV-2 rapid antigen test. This version includes plastic insert components (e.g., 132a, 132b) disposed between an upper portion and lower portion. These insert components 132a, 132b can be injection molded and contain paper and/or glass fiber layers (e.g., leukocytes filter, reagent-treated lysing material, wicking material, etc.) without requiring adhesive to hold them all together. As shown, the wicking channel 111 is roughly teardrop shaped, as opposed to the more rectangular shaped channel 11 shown in FIG. 2. In some examples, the wicking material includes a glass microfiber material infused with a lysing agent, which performs both lysing and wicking for enhanced plasma separation efficiency. Also as shown, the pull tab 120 has a single control arm that combines the functions of the separate control arms shown in FIG. 4. The pull tab 120 also includes guide rails extending downward into guide channels in the lower portion of the housing 102 to help mitigate against the pull tab 120 becoming skewed as it is withdrawn from the housing 102. As shown, an absorbent material, such as a towelette 134 is disposed within the containment chamber 117 to absorb unwanted substances that may result from processing the sample 17. The lower portion may also include a sample-collection region 136 configured to receive a sample-collection device, such as an assay microplate, to facilitate transfer of the extracted sample to an external testing device, reducing sample manipulation that could lead to contamination.

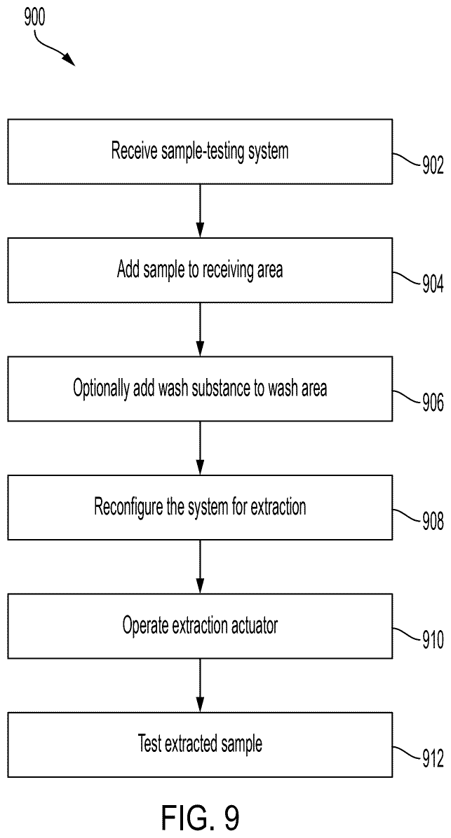

FIG. 9 shows a flowchart 900 for an example method of testing a biological sample. At step 902, the method includes receiving a sample-testing system 100 such as one or more of the embodiments discussed above. At step 904, the method includes adding a sample (e.g., a biological sample) to the sample-receiving area 104 of the sample-testing system 100. As discussed above, a biological sample may include blood sample, a plasma sample, a serum sample, a sputum sample, a urine sample, a saliva sample, a buccal mouthwash sample, a tissue sample, fecal matter, sweat, spinal fluid, amniotic fluid, interstitial fluid, tear fluid, or bone marrow, and so forth. In one example, adding a sample includes adding about 100 microliters of blood to the sample-receiving area 104. At step 906, the method may include (e.g., after waiting about five minutes) adding one or more substances to the wash-receiving area 113. For example, step 906 may include adding about 500 microliters of a first wash substance that is configured to prime the capture pad for RNA capture. Step 906 may also include adding about 500 microliters of a second wash substance to the wash-receiving area 113, the second wash substance configured to flush remaining lysed components through the capture pad and into the sample containment portion of the housing 102.

At step 908, the method includes reconfiguring the sample-testing system 100 into the second configuration (e.g., the sample-extraction configuration). In some examples, reconfiguring the sample-testing system 100 includes operating a reconfiguration mechanism 120 to reconfigure the housing 102 from the first configuration to the second configuration. For example, the reconfiguration mechanism 120 may be a pull tab 120, and operating the pull tab 120 may include pulling the pull tab 120 away from the housing 102 until the pull tab 120 is fully extended. In embodiment where one or more ratchets, step 908 may include pulling the pull tab 120 past each ratcheting mechanism. Step 908 may include listening for a click as the pull tab 120 is pulled past each ratchet into its fully extended position.

At step 910, the method includes operating the extraction actuator 106, causing the component of the sample to be extracted. In some examples, the extraction actuator 106 includes a single-use pushbutton 106, and operating the extraction actuator 106 includes pressing the single-use pushbutton 106. In some examples, pressing the single-use pushbutton 106 causes thin breakaway tabs to break away under the pressure as the single-use pushbutton 106 is depressed. At step 912, the method optionally includes testing the extracted sample. In some examples, the sample-testing system 100 includes an integrated and/or internal assay device or system configured to test the extracted sample, and testing the sample includes extracting the sample into the assay device for testing.

In some examples, the sample-testing system 100 includes an external assay device or system, and testing the sample includes transferring the sample into the assay device for testing. An external assay device may include a loop-mediated isothermal amplification (LAMP) system and/or a PCR thermocycler to amplify the genetic material within the extracted sample. Testing the sample may include adding a dye, such as an asymmetrical cyanine dye to the amplified sample to perform a colorimetric assay. In some examples, adding a dye includes adding a dye such as SYBR Green that binds to specific genetic material. In some examples, testing the sample includes adding a nucleic acid probe, such as a (fluorescently labeled) fragment of complementary genetic material.

A prototype sample-testing system 100 was tested using whole blood samples (at a hematocrit of 40%) doped with different concentrations of HIV viruses. The blood samples were added to the sample-receiving area 104 well of the sample-testing system 100. The plasma was separated, the viruses were lysed, and the RNA was captured on a chitosan-functionalized fusion 5 (FF5) membrane. At least a portion of the FF5 membrane was extracted into a PCR tube containing loop-mediated isothermal amplification (LAMP) reagents and heated at 65° C. for 30 minutes. 1 uL of colorimetric dye, SYBR Green I (2500X) was added to the reaction after 30 minutes. Because of the dye, if the LAMP process formed double stranded DNA (dsDNA) products due to the presence of trigger (HIV RNA), the reaction would turn green. If not, the reaction would stay orange.

Concentrations of 10,000 to 1,000 HIV RNA copies/mL were tested (as well as a baseline of 0 copies/mL to test for false positive results). Each non-0 concentration was tested five separate times, and the baseline test was performed ten times. No false negatives were detected, but one false positive was detected. Therefore, the limit of detection (LOD) of the sample-testing system 100 appears to be 1,000 HIV RNA copies/mL or less.

While the invention has been described with specific embodiments, other alternatives, modifications and variations will be apparent to those skilled in the art. Accordingly, it will be intended to include all such alternatives, modifications, and variations within the spirit and scope of the appended claims.

1. A system comprising:
a separation system configured to separate a component of a sample for testing; and
a housing enclosing the separation system, the housing comprising:
a sample-receiving area configured to, when the housing is in a first configuration, receive the sample and direct the sample to the separation system, causing the separation system to separate the component of the sample;
an extraction actuator configured to, when the housing is in a second configuration, extract the component of the sample from the separation system;
wherein, when the housing is in the first configuration, the extraction actuator is configured to not extract the component of the sample; and
a reconfiguration mechanism operable to reconfigure the housing from the first configuration to the second configuration.

2. The system of claim 1, wherein, when the housing is in the second configuration, the reconfiguration mechanism is inoperable.

3. The system of claim 1, wherein the separation system is configured to separate and/or isolate nucleic acids.

4. The system of claim 1, wherein the reconfiguration mechanism comprises a pull tab, wherein the pull tab comprises a locking mechanism that prevents the pull tab from reconfiguring the housing when the housing is in the second configuration.

5. The system of claim 4, wherein the locking mechanism comprises a ratchet.

6. The system of claim 1, wherein the extraction actuator is configured to be inoperable after the component of the sample is extracted.

7. The system of claim 1, wherein the extraction actuator is a pushbutton configured for a single use.

8. The system of claim 1, wherein:
the separation system comprises a capture material; and
the extraction actuator is configured to extract the component of the sample from the separation system by extracting some or all of the capture material.

9. The system of claim 8, wherein the reconfiguration mechanism is configured to align the some or all of the capture material with the extraction actuator when the housing is in the second configuration.

10. The system of claim 9, wherein the extraction actuator is not affixed to the reconfiguration mechanism.

11. The system of claim 1, wherein the housing further comprises a wash-receiving area configured to, when the housing is in the first configuration, receive a wash substance and direct the wash substance to the separation system.

12. The system of claim 1, wherein the sample-receiving area comprises a basin.

13. The system of claim 11, wherein an interior of the housing defines a containment chamber configured to collect unwanted components of the sample and/or the wash substance.

14. A method comprising:
receiving the system of claim 1 in the first configuration;
adding the sample to the sample-receiving area;
reconfiguring the system into the second configuration; and
operating the extraction actuator, causing the component of the sample to be extracted.

15. The method of claim 14, further comprising, before reconfiguring the system into the second configuration, adding a wash substance to a wash-receiving area.

16. The method of claim 14, further comprising testing the component of the sample.

17. A kit comprising: (i) a system of claim 1; (ii) a collector configured to collect the sample; and (iii) a container configured to receive the component.

18. The kit of claim 17, wherein the kit further comprises one or more of a washing buffer, a nucleic acid probe, and a reaction solution.

19. The system of claim 1, wherein the sample-receiving area is configured to receive a blood sample, a plasma sample, a serum sample, a sputum sample, a urine sample, a saliva sample, a buccal mouthwash sample, a tissue sample, fecal matter, sweat, spinal fluid, amniotic fluid, interstitial fluid, tear fluid, or bone marrow.

20. A system comprising:
a separation system configured to separate a component of a sample for testing;
a housing enclosing the separation system, the housing comprising:
a sample-receiving area configured to, when the housing is in a first configuration, receive the sample and direct the sample to the separation system, causing the separation system to separate the component of the sample; and
a reconfiguration mechanism configured to reconfigure the housing from the first configuration to a second configuration; and
an assay system configured to, when the housing is in the second configuration:
receive the component of the sample; and
perform an assay on the component of the sample.

21. A housing configured to hold a separation system for separating a component of a sample for testing according to claim 1.
